(19) United States
(12) Patent Application Publication
(10) Pub. No.: US 2006/0173362 A1
(43) Pub. Date: Aug. 3, 2006

(54) Methods of medical imaging using quantum dots

(75) Inventors: Steven A. Toms, Shaker Heights, OH (US); Wei-Chiang Lin, Miami, FL (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation and Vanderbilt University

(21) Appl. No.: 11/246,573

(22) Filed: Oct. 7, 2005

Related U.S. Application Data
(60) Provisional application No. 60/617,534, filed on Oct. 8, 2004.

(51) Int. Cl. A61B 6/00

(52) U.S. Cl. 600/478

(57) ABSTRACT

A method for identifying cells of a living subject in vivo includes delivering a plurality of optical nanoparticles to the cells, optically imaging the optical nanoparticles, and identifying the cells of a living subject from an image and/or spectrum of the optical nanoparticles.

### RELATED APPLICATIONS

The present application claims priority from U.S. Provisional Application No. 60/617,534, filed Oct. 8, 2004 herein incorporated by reference in its entirety.

### FIELD OF THE INVENTION

The present invention relate to a method and system of medical imaging, and, more particularly, to a method and system of medical imaging using quantum dots.

### BACKGROUND OF THE INVENTION

Optical imaging of biological and/or chemical conditions of tissues of a living subject is an area of significant scientific and clinical importance. Typically, conventional optical labels such as fluorescent dyes are introduced into tissue samples to signal changing biological and/or chemical conditions of tissues of a living subject. Despite many successful applications, conventional optical labels have many drawbacks. For example, conventional optical labels are general toxic to living cells and tissues comprised of living cells. Additionally, conventional optical labels such as fluorescent dyes generally suffer from short-lived fluorescence because the dyes undergo photo bleaching after minutes of exposure to an excitation light source. This renders them unsuitable for optical imaging that requires extended time period of monitoring. Moreover, conventional optical labels are sensitive to environmental changes such as pH and oxygen concentration.

Another drawback of conventional optical labels is that, typically, the excitation spectra of such labels are quite narrow, while the emission spectra of such labels is relatively broad, resulting in overlapping emission spectra. Thus, when a combination of conventional optical labels with different emission spectra are used in optical imaging, multiple filters are need to detect the resultant emission spectra of the combination.

### SUMMARY OF THE INVENTION

The present invention relates to a method of identifying cells of a living subject in vivo. In the method, a plurality of optical nanoparticles can be delivered to the cells. The optical nanoparticles can be illuminated with an excitation light at wavelength effective to make the optical nanoparticles emit a detectable wavelength of light. The light emitted by the optical nanoparticles can be detected to provide an image and/or spectrum of cells being detected.

In aspect of the invention, the optical nanoparticles can comprise a plurality of quantum dots. The quantum dots can be water soluble and comprises a core and a cap. The core of the quantum dot can be metallic and/or semiconductive. The delivering step can comprise intravenously administering the plurality of quantum dots to the subject being treated at a threshold concentration effective to readily detect the cells upon excitation with the excitation light. The excitation light can comprises a coherent light and/or a non-coherent light.

Another aspect of the invention relates to a surgical method or procedure of imaging tissue of a living subject in vivo. In the method, an effective amount of optical nanonparticles can be preoperatively delivered to the subject being treated. The optical nanoparticles can be detected in the subject with an optical device. A surgical procedure on the subject being treated can be guided with the detected optical nanoparticles.

Examples of the surgical procedures can include a biopsy, a surgical excision, a laser ablation or a radiofrequency ablation. In one aspect, the step of detecting the quantum dots can comprise a step of imaging the optical nanoparticles preoperatively and/or intra-operatively. The method can further comprise a step of imaging the optical nanoparticles post-operatively for assessing the performance of the surgical procedure.

In another aspect, the step of imaging the optical nanoparticles can comprise at least one of imaging technique that can include optically imaging the optical nanoparticles, utilizing ultrasound to image the optical nanoparticles, utilizing computed tomography (CT) to image the optical nanoparticles, and/or utilizing magnetic resonance imaging (MRI) to image the optical nanoparticles.

In another aspect, the present invention relates to a method of imaging a neoplasm (e.g., tumor) of a living subject in vivo for image guided surgery. In the method, an effective amount of optical nanoparticles can be preoperatively delivered to tissues associated with the neoplasm. The optical nanoparticles can be detected in the subject with an optical device. A surgical procedure on the tumor can be guided with the detected quantum dots. The neoplasm can comprise a tumor (e.g., brain tumor) and/or other abnormal tissues.

Yet another aspect of the invention relates to a method for identifying a cause for a medical condition of a living subject. In one embodiment, the method includes the steps of conjugating a plurality of optical nanoparticles with an antibody responsive to the medical condition. An effective amount of the optical nanoparticles with the antibody can be delivered to the circulation system of the living subject. The optical nanoparticles can be optically imaged and the cause of the medical condition can be determined from the images of the optical nanoparticles with the antibody.

In an aspect of the invention the optical nanoparticles can comprise a plurality of quantum dots with different wavelengths, and the antibody conjugated with quantum dots with a predetermined wavelength. The images of the quantum dots with the antibody comprise a spectrum with corresponding wavelength quantum dots. In one embodiment, the determining step comprises a step of identifying the predetermined wavelength from the spectrum so as to determine the organism for the medical condition. In one embodiment, the medical condition relates to an infection. In another embodiment, the medical condition relates to an inflammation.

In yet another aspect, the present invention relates to a method for using quantum dots as contrast agent for imaging at least a part of a living subject. The invention includes the steps of loading an electron dense material into a quantum dot shell, delivering an effective amount of quantum dots loaded with the electron dense material to the part of the living subject, optically imaging the part of the living subject, and obtaining medical information regarding the living subject from the optical images of the living subject.

In one aspect, the step of delivering comprises the steps of conjugating the quantum dots with peptides and nucleic acids and administering the mixture to the living subject. The effective amount of quantum dots delivered is no less than a threshold concentration of quantum dots. In one embodiment, the step of imaging comprises a step of utilizing x-ray to image the part of the living subject. In another aspect, the step of imaging comprises a step of utilizing computed tomography to image the part of the living subject. In yet another aspect, the step of imaging comprises a step of utilizing angiography to image the part of the living subject. In one aspect, the part of the living subject is related to an organ of the living subject.

The present invention further relates to a method of detection of a substance in a sample. In one aspect, the method includes the steps of adding a plurality of optical nanoparticles to the sample, waiting for a period of time to allow the plurality of optical nanoparticles to bind to the substance in the sample to form an optical nanoparticle-substance mixture, removing optical nanoparticles that do not bind to the substance in the sample, illuminating the optical nanoparticle-substance mixture in the sample with an excitation light, collecting light returned from the illuminated optical nanoparticle-substance mixture in the sample, obtaining an image and/or spectrum of the optical nanoparticle substance mixture in the sample from the collected light, and detecting the substance from the image and/or spectrum of the optical nanoparticle-substance mixture.

The invention further comprises the step of conjugating the plurality of optical nanoparticle with a predetermined chemical compound. The predetermined chemical compound can comprises a protein, a peptide, a nucleic acid, or an antibody. Additionally, the invention comprises the steps of finding a sign of pathogenesis from the image and/or spectrum of the optical nanoparticle-substance mixture in the sample and identifying a corresponding disease from the identified sign of pathogenesis the plurality of optical nanoparticles with a predetermined chemical compound. Furthermore, the present invention comprises the steps of identifying the peak of the quantum dot-substance mixture in the spectrum at a predetermined wavelength and using the peak of the optical nanoparticle-substance mixture at the predetermined wavelength as the sign of pathogenesis to identify the disease.

In one aspect, the adding step comprises a step of adding the plurality of optical nanoparticles with a concentration to the sample. The concentration of the plurality of optical nanoparticles is no less than a threshold concentration. The length of the waiting time in the waiting step is related to the concentration of optical nanoparticle-substance mixture.

In one aspect, the excitation light comprises a coherent light. In another embodiment, the excitation light comprises a non-coherent light. The collecting step comprises the step of collecting light returned from the illuminated optical nanoparticle-substance mixture to an optical spectroscopic system or long-pass filter system. The obtaining step comprises the step of obtaining an image and/or spectrum of the illuminated quantum dot-substance mixture.

In one aspect, the sample comprises a liquid sample of at least one of cerebrospinal fluid, thoracentesis, paracentesis, saliva, urine, semen, mucous, blood, and bronchoalveolar specimen of a living subject. In another aspect, the sample comprises a solid sample of at least one of stool, pap smear, and buccal membrane scrapings of a living subject.

In one aspect, the substance relates to a sign of pathogenesis including antigen, peptide sequence, nucleic acid sequence, RNA or DNA of an infectious pathogen such as viral, bacterial, or parasitic pathogen, and cancer cells. The sign of pathogenesis is corresponding to a disease related to infection, inflammation, or cancer.

In another aspect, the present invention relates to a device for detection of a substance in a sample. The device includes a plurality of optical nanoparticles, a spectra containing a plurality of spectrum, each spectrum corresponding to a corresponding optical nanoparticle-substance mixture, and an optical device for obtaining an image and/or spectrum of an optical nanoparticle-substance mixture in a sample. In operation, a substance is identifiable from comparison the image and/or spectrum of the optical nanoparticle-substance mixture in the sample with the spectra.

In yet another aspect, the present invention relates to a method for detecting a therapeutic agent delivery. In the method, the therapeutic agent can be labeled with an effective amount of nanoparticles. The therapeutic agent with an effective amount of nanoparticles can be delivered to a targeted site of the living subject. The targeted site of the living subject can be imaged, and the delivery of the therapeutic agent can be detected from the image of the targeted site of the living subject.

In one aspect, the delivery is effective when the image of the targeted site of the living subject contains the image of at least some of the optical nanoparticles. The optical nanoparticles can comprise at least one of quantum dots and/or ultrasmall paramagnetic iron oxide particles (USPIO). The ultrasmall paramagnetic iron oxide particles comprise a collection of iron oxide particles with enclosures such as silica or dextran.

In one aspect, the therapeutic agent comprises a therapeutic cell that comprises at least one of stem cells, package delivery cells carrying a packaged gene or protein, and immunotherapeutic cells. The labeling step comprises the step of labeling the therapeutic cell by delivering an effective amount of nanoparticles to the cell. In another aspect, the therapeutic agent comprises a therapeutic gene. The labeling step comprises the step of labeling the therapeutic gene by conjugating the optical nanoparticles to the therapeutic gene. The delivering step comprises the step of delivering the therapeutic agent labeled with the optical nanoparticles intravenously.

In one aspect, the step of imaging comprises at least one of optically imaging the targeted site of the living subject, utilizing ultrasound to image the targeted site of the living subject, utilizing computed tomography to image the targeted site of the living subject, utilizing magnetic resonance imaging to image the targeted site of the living subject.

### BRIEF DESCRIPTION OF THE DRAWINGS

The foregoing and other aspects of the present invention will become apparent to those skilled in the art to which the present invention relates upon reading the following description with reference to the accompanying drawings.

FIG. 1 illustrates a magnetic resonance image (MRI) of a rat brain about 2 to 3 weeks after implantation with a tumor.

FIG. 2 illustrates an image of a rat brain infused with quantum dots in accordance with an aspect of the invention.

FIG. 3 illustrates optical spectroscopy of a rat liver 24 hours after infusion with quantum dots in accordance with an aspect of the present invention.

FIG. 4 illustrates a magnetic resonant image of a rat brain infused with quantum dots.

FIG. 5 illustrates UV microscopy of rat brain after infusion with quantum dots in accordance with an aspect of the invention.

FIG. 6 is a schematic illustration of an optical device in accordance with the present invention.

FIG. 7 illustrates optical spectroscopy of quantum dots in solution accordance with an aspect of the invention.

FIG. 8 illustrates optical spectroscopy of quantum dots in solution in accordance with an aspect of the invention.

FIG. 9 illustrates optical spectroscopy of quantum dots in solution in accordance with an aspect of the invention.

FIG. 10 illustrates in vivo optical spectroscopy of quantum dots in a rat liver in accordance with an aspect of the invention.

FIG. 11 illustrates low magnification optical imaging (8-25×) of rat liver infused with quantum dots in accordance with an aspect of the invention.

FIG. 12 illustrates low magnification optical imaging (8-25×) of rat spleen infused with quantum dots in accordance with an aspect of the invention.

FIG. 13 illustrates low magnification optical imaging (8-25×) of rat lymph nodes infused with quantum dots in accordance with an aspect of the invention.

FIG. 14 illustrates xenogen IVIS fluorescence imaging of control rat brain in accordance with an aspect of the invention.

FIG. 15 illustrates xenogen IVIS fluorescence imaging of experimental gliomas of a rat brain in accordance with an aspect of the invention.

FIG. 16 illustrates xenogen IVIS fluorescence imaging of another experimental gliomas with lesions on the surface of the rat brain in accordance with an aspect of the invention.

FIG. 17 illustrates hematoxylin and eosin (H&E) analysis of the frontal tumor of FIG. 16.

FIG. 18 illustrates fluorescence imaging of brain surface at 20× showing quantum dots on surface of the primary tumor from FIG. 16.

FIG. 19 illustrates UV photomicroscopy of quantum dots on the borders of tumors.

FIG. 20 illustrates confocal microscopy of GFAP labeled astrocytes.

FIG. 21 illustrates quantum dots co-localize with CD11b positive macrophages and microglia.

### DETAILED DESCRIPTION

The present invention relates to a method of identifying cells or tissue of a living subject in vivo using optical nanoparticles. By optical nanoparticles, it is meant aggregates of anywhere from a few hundred to tens of thousands of atoms that combine into a form with a diameter ranging from about 3 about 25 nanometers and that are capable of being excited with a wavelength of light resulting in detectable light emissions (e.g., fluorescence emissions) of high yield, and with discrete peaks, for example, from the ultra-violet to infrared wavelength light spectrum.

The optical nanoparticles can include quantum dots or nanocrystals. Quantum dots and nanocrystals, which are used interchangeably throughout the specification and claims, refers to semiconductor nanocrystals that can be excited with a single wavelength of light resulting in detectable fluorescence emissions of high quantum yield, and with discrete fluorescence peaks (e.g. having a narrow spectral band such as between about 10 nm to about 60 nm). Quantum dots can comprise an optical core that is surrounded by a protective shell. By way of example, the optical core can comprise a cadnium/selenium core and/or a cadium/selenium/telluride core.

The protective shell can comprise a zinc sulfide shell. The protective shell can be modified to promote water solubility and to enhance to enhance stability under complex conditions of aqueous environments encountered in living tissues and in protocols for labeling tissues. For example, the protective shell can be conjugated to methoxy terminated polyethylene glycol amine to improve the circulation half-life of the quantum dot and mitigate non-specific uptake by the reticuloendothelial system (RES). Quantum dots comprising a cadnium/selenium core, a zinc sulfide protective, and a conjugated 500 Dalton amino polyethylene glycol polymer (PEG NH₂ quantum dots) are commercially available from Quantum Dot Corporation, Hayward, Calif. These quantum dots are capable emitting light at a wavelength of about 705 nm (±about 20 nm) upon excitation of about with light having a wavelength of about 400 nm ((±about 40 nm).

The quantum dots used in accordance with the method of invention can also be substantially non-toxic to living tissue (e.g., such that they can be used to label living tissue or cell processes active in living tissue) as well as be sensitive in terms of being detected in fluorescence imaging of tissue because of their fluorescence properties (e.g., including, but not limited to, high quantum efficiency, resistance to photo-bleaching, and stability in complex aqueous environments). Additionally, the quantum dots can be linked to other agents, including chemical compounds, such as proteins, peptides, nucleic acids, and antibodies (e.g., monoclonal and polyclonal antibodies) by conjugation to the protective shell.

Other optical nanoparticles that can be used in the present invention can include inorganic materials, such as ultrasmall super paramagnetic iron oxide (USPIO) particles, commercially available under the name Combidex, from Advanced Magnetics Corporation, Cambridge, Mass. Ultrasmall paramagnetic iron oxide particles can comprise a collection of iron oxide particles with enclosures such as silica or dextran. It will be that other optical nanoparticles, besides quantum dot can used in accordance with the present invention. These other optical nanoparticles can include organic materials, such as light (infrared) absorbing and emitting episomes.

The optical nanoparticles in accordance with the present invention can be used in in vivo intra-operative methods where detection and imaging of cells or tissue cannot readily be performed with traditional optical detection or imaging techniques. These methods can include, for example, surgical navigation and guidance, endoscopic navigation and guidance in the aerodigestive track, endovascular detection of ruptured atherosclerotic plaques and monitoring in indwelling endovascular devices, cancer imaging, infection or inflammation imaging, imaging of cell and tissue apotosis, localization of neurologic pathways involved in chronic pain, and localization of epilepsy foci. It will be appreciated that the optical nanoparticles can be used in other in vivo methods as well as intraoperative procedures.

In each method, a plurality of the optical nanoparticles (e.g., quantum dots) can be delivered to the cells or tissue of the subject in vivo by administering an effective amount or concentration of the optical nanoparticles to the subject. By effective amount or concentration of optical nanoparticles it is meant an amount of optical nanoparticles that when excited with an appropriate excitation wavelength, will emit fluorescence emission wavelength sufficient for detecting and imaging the target cells or tissue. As apparent to one skilled in the art, such an amount will vary depending on factors that include the amount of tissue to be imaged, the rate of contact of the optical nanoparticles with the tissue, any abnormalities of the tissue that may affect the efficiency of the optical nanoparticles contacting or binding to the tissue.

In an aspect of the invention, the optical nanoparticles can be administered to the subject by venous (or arterial) infusion. In venous infusion, an effective amount or concentration of optical nanoparticles administered to subject can be that amount or concentration that is detectable in the tissue or cells after sequestration of the optical nanoparticles in the liver, spleen, and lymph nodes. Optionally, the optical nanoparticles can be administered to the subject by directly injecting the optical nanoparticles into cells or tissue of the area being identified or an area proximate or peripheral to the area being identified. Direct injection of the optical nanoparticles can be performed by using, for example, a syringe.

The optical nano-particles upon administration to the subject can localize to the cells or tissue of being identified. The localization of optical nanoparticles will depend on the specific optical nanoparticle used in the method of the invention. For example, it has been found that optical nanoparticles, such as quantum dots (e.g., PEG NH₂ quantum dots) upon administration of an effective amount to the subject can be taken up by macrophages that co-localize to peripheral areas (i.e., peripheral tissue) of inflamed and/or neoplastic tissue. Alternatively, the nanoparticle can be conjugated to chemical compounds, such as a protein, a peptide, a nucleic acid, or antibody that is specific to the cell type or tissue type. Upon administration of the conjugated optical nanoparticle to the subject, the optical nanoparticle can localize to the cells or tissue specific to the conjugates. For example, quantum dots can be conjugated to herceptin, which can target Her2-Neu positive breast cancer.

The optical nanoparticles localized to the cells or tissue can then be detected to provide an image and/or spectrum of detected cells. In an aspect of the invention, the optical nanoparticles can be imaged using optical spectroscopy and/or optical imaging techniques, such as specialized lighting and goggles, UV microscopy, and/or charge coupled device (CCD) cameras. In these techniques, the optical nanoparticles can be illuminated with an excitation light at a wavelength that causes the optical nanoparticles to emit a detectable wavelength of light. The light emitted by the optical nanoparticles can then be detected to provide an image and/or spectrum of cells being detected.

In an aspect of the invention, the excitation light can comprise, for example, a UV light from sources, such as UV lamp, nitrogen laser source, or blue light with an emission wavelength of about 250 nm to about 400 nm. The excitation light can be delivered to the tissue site of detection with a fiberoptic system, which also collects the light from the illuminated optical nanoparticles at the tissue site of detection. The collected light can be fed into a detector to record emission light, for example, in the about 300 nm to about 2000 nm wavelength range. The recorded emission light in the about 300 nm to about 2000 nm range can be processed into spectroscopic data to illustrate the condition (i.e., location) of the cells or tissue at the site of detection.

Light delivery to and collection from the optical nanoparticles can be performed using a number of commercially available components. For example, a high-pressure nitrogen laser (337 nm, Oriel Corp. Stratford, Conn.) can be used as the excitation source for fluorescence measurements. Light delivery and collection can also be achieved with a “Gaser” fiberoptic probe (Visionex, Inc. Atlanta, Ga.). This probe comprises seven 300 micron-diameter fibers with a central fiber and surrounding fibers. The central fiber can be directed conventionally and the tips of the surrounding fibers can be tapered to optimize overlap of excitation and collection volumes. Two of the surrounding fibers can deliver light pulses to the tissue sample while the remaining fibers collect fluorescence emission from the tissue sample sequentially. The collected signal can then be dispersed with a spectrograph (Triax 180, Instruments S. A., Inc., Edison, N.J.) and detected with a thermoelectrically cooled CCD camera (Spectra One, Instruments S. A., Inc., Edison, N.J.).

For fluorescence measurements, reflected laser light can be eliminated with two 360 nm long pass filters placed in front of entrance slit of the spectrograph. The light collected by the probe can be dispersed with a spectrograph (Triax 180, Instruments S. A., Inc., Edison, N.J.), which includes a 300 gr/mm grating and detected by a thermoelectrically cooled CCD camera (2000×800 pixels, Spectra One, Instruments S. A., Inc., Edison, N.J.). The entire system can be computer controlled. A medical grade isolation transformer (ITI 500-4, Dale Technology Corp. New York) can be used with the portable spectroscopic system to comply with the electrical safety standard of the operating room.

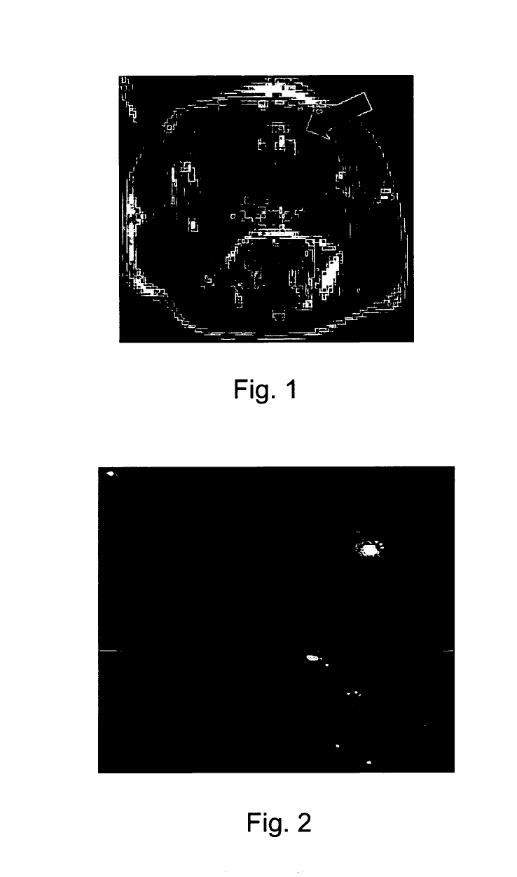

In accordance with one example of the method, water soluble PEG-NH₂ 705 nm quantum dots, can be co-localized with brain tumors and detected with optical imaging. In this example, rat C6 25 glioma cell lines were transplanted into nude rats brains and grown. As shown in FIG. 1, a MRI (Magnetic Resonance Image) of a rat brain 2-3 weeks post-implantation shows successful tumor growth indicated by the arrow. Water soluble PEG-NH2 705 nm quantum dots were administered to the rat by intravenous infusion into the rat brain. About 24 to about 72 hours after the infusion, the histologic sections through the tumor region of the rat brain were excited by blue wavelength light and the light emitted was visualized by a charge-coupled device (CCD) camera. The image visualized by CCD camera is shown in FIG. 2. The illuminated areas are water soluble PEG-NH2 705 nm quantum dots co-localized with brain tumors. The identification of quantum dots can enable guided and more complete surgical resections of brain tumors.

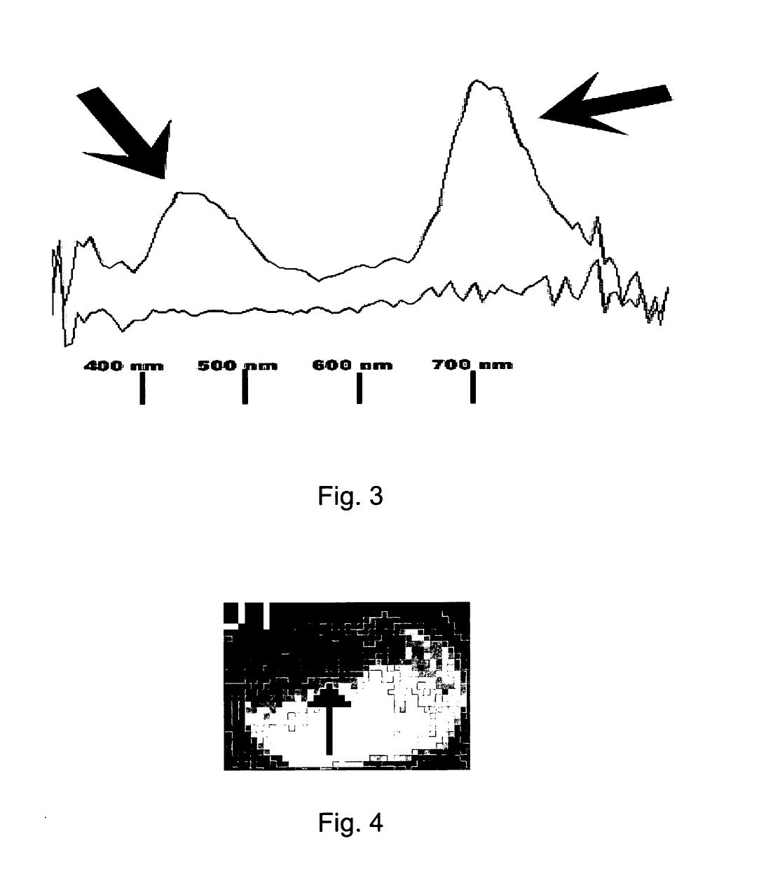

In accordance with another example, water soluble PEG-NH2 705 nm quantum dots were injected into the liver of a nude rat. About 24 hours after the injection, the spectroscopic data of the water soluble PEG-NH2 705 nm quantum dots were collected and process and the results shown in FIG. 3. The lower line is the baseline spectrum, measuring ambient light conditions during the test. The upper line is the test spectrum. The tissue auto fluorescence peak is at about 460 run emissions. The quantum dots fluorescence has a 705 nm emission peak 240.

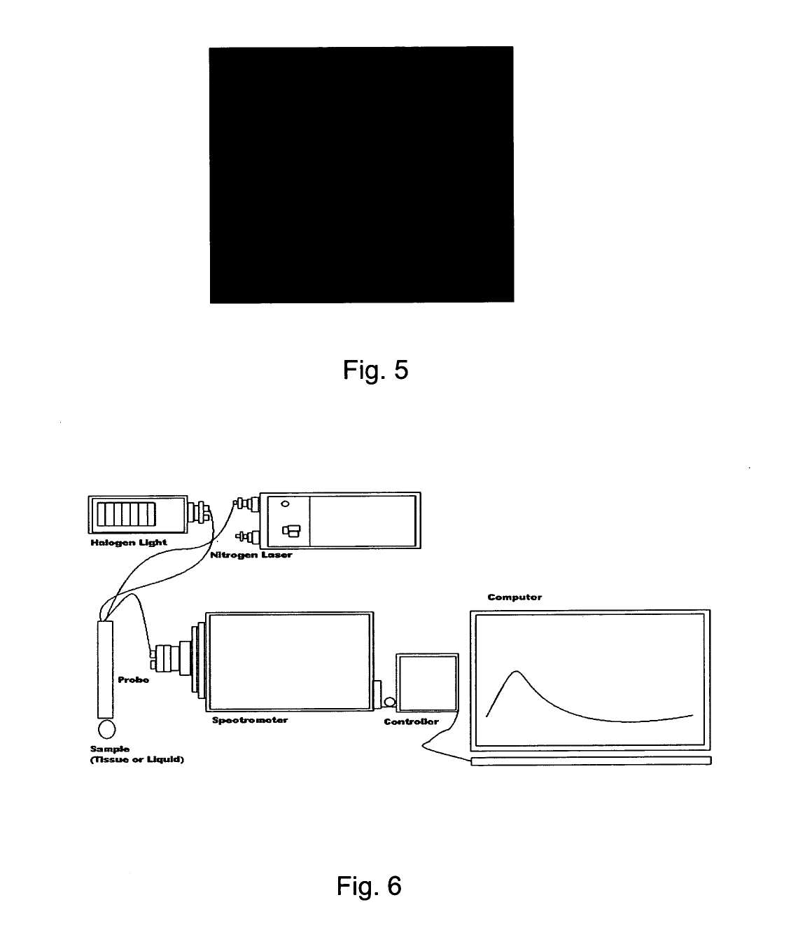

It will be appreciated that although optical nanoparticles (e.g., quantum dots) can be detected with optical imaging or spectroscopy methods, it is also possible detect optical nanoparticles using other methods, such as magnetic resonance imaging (MRI) and x-ray. MRI relies upon changes in magnetic dipoles to perform detailed anatomic imaging and functional studies. Preliminary data developed with quantum dots suggests that interstitial infusion of quantum dots into rat brain is visible on MRI as loss of signal. For example, FIG. 4 is a MRI image of a rat brain. Arrow points to a darker area of signal loss secondary to magnetic susceptibility artifact caused by interstitial infusion of water soluble PEGNH2 705 nm quantum dots into the rat brain. The darker area pointed by arrow is in direct contrast to the white and light gray colors of the normal, contra lateral hemisphere, respectively. Referring to FIG. 5, UV microscopy of tissues from this region of the brain shows a sea of red fluorescence from the water soluble PEG-NH₂ 705 nm quantum dots against the background blue tissue auto fluorescence.

The electron dense core of optical nanoparticles, such as quantum dots and USPIOs, can also make them highly visible on X-ray, monochromatic X-ray, computed tomography (CT) and ultrasound (US).

Monochromatic x-rays sources have the ability to generate X-ray photons 18 pico seconds pulse throughout its tunable 12-50 keV range. These electrons have the ability to be useful in imaging as well as to deliver therapeutic irradiation. A prototype terawatt IR laser has been developed by Myxis Corp in Nashville, Tenn. (http://www.mxisvstems.com) and is being studied for imaging and therapeutic applications. Among these applications are k-edge imaging and Auger cascade electron generation for cancer therapeutics. K-edge refers to the specific binding energy of the innermost or k-orbit electron in the atom of interest. If, for example, iodine was introduced into a tissue by means of an iodine tagged tumor affinitive drug, one could detect its presence in rather small concentrations given a monochromatic beam tuned to 33.2 keV (the binding energy of its k-orbit electron) and a good imaging detector. The X-ray photon, in this case, matches when the k-orbit electron of iodine is knocked out of its orbit by tuning monochromatic X-rays to 33.2 keV. The ejected electron is replaced in the K orbit by an electron from the L-orbit. As this electron drops from the L to the K-orbit, it gives off a 28.3 keV characteristic photon. Likewise, the L-orbit electron is then replaced by an electron from the M-orbit. This in turn gives off a 4.3 keV photon. The N-orbit follows the lead of the other shells and contributes an electron to the M orbit giving off a 0.6 keV 5 photon. Adding up the energies of these various photons comes to 33.2 keV. These characteristic photons interact with the matter in their surrounding medium, traveling less then a few microns at most for the softer X-rays, but penetrating some distances for the more energetic ones. This entire process is known as an Auger cascade.

Cadmium in the optical nanoparticles, such as quantum dots, has a k-orbit energy of 29.3 keV, which is well within the range of the tunable monochromatic laser. Quantum dots appear to be biologically inert. When coupled to oligonucleotides or peptides that bind steroid receptors, the quantum dots have the potential to bind nuclear DNA and serve as k-orbit electron donors in an Auger irradiation cascade, thus increasing the therapeutic potential of irradiation.

In addition, k-orbit imaging of optical nanoparticles with the monochromatic source would allow for one agent, the quantum, to serve as a contrast agent for k-orbit monochromatic CT imaging as well as optical imaging during surgical interventions. Given that optical nanoparticles may be loaded into therapeutic cells such as stem cells, covalently linked to nucleic acids and proteins, imaged with optical imaging and optical spectroscopy, HI, CT and US.

Optionally, the core and or shell of the optical nanoparticles, such as quantum dots, can be modified to facilitate detection and imaging with MRI and CT as well as positron emission tomography (PET). For MRI applications, gadolinium tags can be attached to the shell and/or iron oxide can be used in the core of the quantum dots. For PET applications, radioactive tags can be attached to the shell of the quantum dots. For CT applications, iodide or other heavy metals can be attached to the shell and/or core of the quantum dots to facilitate CT contrast.

It will be appreciated the optical nanoparticles in accordance with the present invention will likely be most useful clinically when several imaging techniques or imaging followed by a medical or surgical procedure is used. In this way, the ability to use one agent for multiple imaging modalities is optimized making the optical nanoparticles cost-competitive with existing contrast agents.

In accordance with an aspect of the invention, the in vivo detection method of the present invention can be used for surgical navigation and guidance in a surgical procedure or method. Examples of the surgical procedures include a biopsy, surgical excision, laser ablation, or radiofrequency ablation.

In the surgical method, an effective amount of the optical nanoparticles (e.g., quantum dots) can be preoperatively delivered to the subject being treated, by for example intravenous infusion or direct injection. The optical nanoparticles can include, for example, quantum dots, such as PEG NH₂ quantum dots. PEG NH₂ quantum dots upon administration to the subject can be readily taken up by macrophages that co-localize to peripheral areas (i.e., peripheral tissue) of inflamed and/or neoplastic tissue. Optionally, the nanoparticles can be conjugated to a chemical compound, such as a protein, nucleic acid, or antibody that is specific to cell type or tissue on which the surgical procedure is performed.

The optical nanoparticles administered to the subject can be detected and imaged in the subject with, for example, optical imaging, such as specialized lighting and goggles, UV microscopy, and/or charge coupled device (CCD) cameras. In these techniques, the optical nanoparticles can be illuminated with an excitation light at a wavelength that causes the optical nanoparticles to emit a detectable wavelength of light. The light emitted by the optical nanoparticles can be then be detected to provide an image and/or spectrum of cells being detected. Light delivery and collection can be performed using a single optical device (e.g., probe) that is inserted into the subject.

The step of detecting the optical nanoparticles can comprise a step of imaging the quantum dots preoperatively and/or intra-operatively. The method can further comprise a step of imaging the quantum dots post-operatively for assessing the performance of the surgical procedure.

The surgical procedure on the subject being treated can be guided with the detected optical nanoparticles (e.g., quantum dots). For example, where the cells or tissue is a neoplasm, such as a brain tumor, the optical nanoparticles administered to the subject can localize about the periphery of the neoplasm via phagocytosis of the quantum dots by perivascular or tissue macrophages congregating about the neoplasm. The optical nanoparticles can then be imaged using intra-operative optical imaging techniques and serve as optical beacons to determine the specific location of the tumor. When the optical nanoparticles are detected, surgical excision, biopsy, or resection of the neoplasm (e.g., brain tumor) can be guided by the optical imaging. This allows the surgeon to more readily locate the neoplasm and discriminate intraoperatively neoplastic and normal tissue. This also provides a safer, more complete, and rapid identification of neoplastic tissue during biopsy, excision, and/or resection of neoplastic tissue.

For multimodal imaging applications, the optical nanoparticles can be administered to the subject and then preoperatively imaged using, for example, CT or MRI. After preoperative imaging, the optical nanoparticles can serve as optical beacons for use during surgery leading to more complete resections or more accurate biopsies. In surgical resection of lesions, the completeness of resection can be assessed with intra-operative ultrasound, CT, or MRI. For example, in glioma (brain tumor) surgery, optical nanoparticles (e.g., quantum dots) can be given intravenously about 24 hours prior to pre-surgical stereotactic localization MRI. The optical nanoparticles can be imaged on gradient echo MRI sequences as a contrast agent that localizes with the glioma. The optical nanoparticles can be visualized optically or with optical spectroscopy during the surgery. Intraoperative MRI (or CT or US) can be used to detect residual optical nanoparticles beyond optical spectroscopy detectability (i.e. quantum dots greater than 1 cm deep into resection cavity).

In prostate cancer lymph node metastasis detection and biopsy, for example, and MRI can be performed 2 days pre-operatively. After the MRI, optical nanoparticles (e.g., quantum dots) can be infused intravenously. A second MRI can then be performed 24 hours after the optical nanoparticle infusion. All lymph nodes that take up the infused optical nanoparticles may show a signal loss on the second MRI. These lymph nodes are likely normal. Areas that show lymph nodes with few optical nanoparticles on MRI are sampled with optical imaging or spectroscopy techniques during surgery. Those without optical nanoparticles (e.g., quantum dots) are likely replaced by cancer and need to be biopsied.

In an aspect of the invention, the optical nanoparticles used in the surgical method can be conjugated to a chemical compound, such as a monoclonal antibody (MAb), peptide, protein, and/or nucleic acid to target tumor specific surface antigens and allow their identification for biopsy, surgical excision, or laser or radiofrequency ablation. More specifically, MAb-quantum dot conjugates, such as epidermal growth factor receptor variant mutant Ab for glioblastoma multiform resection or Herceptin—quantum dot conjugate can be used in image guided surgery. Herceptin targets Her2-Neu. For Her2-Neu positive breast cancer, a needle localization biopsy can place a biopsy needle to its intended target and place an optical spectroscopy probe down to the needle. When the spectrum of the conjugated quantum dot is identified, the target has been localized and a biopsy may be taken. This should improve the yield of biopsy and nearly eliminate negative biopsies. During radical mastectomy, signal from conjugated quantum dots in lymph nodes or soft tissue can represent metastasis tumor, and will therefore aid in completeness of resection.

Optionally, the surgical method can use a first concentration of optical nanoparticles that emit radiation at a first wavelength and a second concentration of optical nanoparticles that emit radiation at a second wavelength different than the first wavelength. The first concentration of optical nanoparticles that emit radiation at a first wavelength can be conjugated to a first chemical compound, such as a monoclonal antibody, peptide, protein, or nucleic acid, that is specific to a first cell or tissue type. The first tissue type can be cells or tissue to be excised, biopsied, or resected, such as cells of a brain tumor. The second concentration of the optical nanoparticles that emit radiation at the second wavelength can be conjugated to a second chemical compound, such as a monoclonal antibody, peptide, protein, or nucleic acid, that is specific to a second tissue type. The second tissue type can be normal tissue that surrounds the first tissue type. Administration of the first concentration of optical nanoparticles and the second concentration of optical nanoparticles to the subject allows the first tissue type (e.g., brain tumor) to be more readily distinguished from the second tissue type (e.g., normal tissue) as each tissue type will emit a distinct optical signature upon excitation. This will allow a surgeon to more readily perform biopsy, surgical excision or resection, or laser or radiofrequency ablation without adversely affecting normal tissue.

In another aspect of the invention, the in vivo detection method of the present invention can be used for endoscopic navigation and guidance in the aerodigestive and genitourinary tracts. In the method, optical nanoparticle (e.g., quantum dot) labeled antibodies specific for cervical carcinoma can be delivered to the cervix of the subject. Antibodies specific to cervical carcinomas can include antibodies to human papilloma virus (HPV), which is thought to be the cause of most cervical cancer. Optical nanoparticle labeled antibodies can be used in coloposcopy to identify cervical epithelium infected by the virus. Any optical nanoparticle positive areas can be biopsied for further analysis. Similarly, Heliobacter infection is thought to underlie a number of gastric ulcers. Administration of optical nanoparticle labeled anti-Heliobacter antibodies prior to endoscopy can identify those patients for whom anti-Heliobacter antibiotic therapy might be indicated.

In a further aspect of the invention, the in vivo detection method of the present invention can be used for endovascular detection of ruptured atherosclerotic plaques and monitoring of indwelling endovascular devices. Detection of ruptured atherosclerotic plaques can be useful in preventing stokes and heart attacks. When these lesions are identified, interventions, such as endovascular stenting, may be performed to reduce the risk of thrombosis or thromboembolism from the ruptured plaque. Optical nanoparticle labeled peptides or antibodies can be delivered intravenously (IV) to allow binding to ruptured plaques. Identification of disrupted vascular endothelium from ruptured plaques during angiography can suggest the need for acute intervention with surgery, stenting, or aggressive medical therapy including anti-platelet agents or anticoagulation.

In a similar fashion, platinum coils introduced to aneurysms or endovascular stents can be coated with optical nanoparticles. After endothelialization takes place, the optical nanoparticles can be less visible to endovascular optical spectroscopy suggesting good healing over the device. Persistence of the spectral features of optical nanoparticles can suggest poor endothelialization or coil extrusion into the vascular lumen, which can in turn suggest the need for further anticoagulation or antiplatelet therapy.

In a further aspect of the invention, the in vivo detection method of the present invention can be used with stem cells for the in vivo detection of gliomas (brain tumors). It is known that stem cells co-localize with infiltrating glioma cells. When mesenchymal or neural stem cells are loaded with optical nanoparticles, infiltrative tumor cells can be imaged by identifying signals of the optical nanoparticles from the stem cells that surround glioma cells infiltrated beyond tumor borders. The imaging can be performed on standard MRIs.

In a further aspect of the invention, the in vivo detection method of the present invention can be used for detection and imaging of areas of inflammation. Areas of inflammation, such as arthritis and demyelination from multiple sclerosis and acute and chronic infections attract white blood cells (WBCs) that mediate inflammatory response. A patient's own WBCs can be collected and labeled using a peptide targeting sequence to facilitate the introduction of optical nanoparticles into WBCs at a high concentration. The cells can then be re-infused intravenously for a localized MRI or optical imaging. This method, in contrast to nuclear medicine tagged white cell studies that rely upon radioactively labeling WBCs and imaging on a Gamma camera, gives more anatomic localization as well as allows the direct comparison with MRIs taken 24 hours prior to intravenous infusion of optical nanoparticle loaded WBCs.

Additionally, more specific identification of the type of bacteria causing an infection may be possible. First, optical nanoparticles (e.g., quantum dot) antibody conjugates with different wavelength optical nanoparticles for each antibody are given intravenously. Then, optical spectroscopic examination of tissues with infection can produce a spectrum indicating the detection of optical nanoparticles with a particular wavelength. Identification of the wavelength can give the type of antibody and identification of the organism responsible for infection. Specific antibiotic therapy can then begin days before traditional bacterial culturing and antibiotic sensitivity testing can be performed.

In yet another aspect of the invention, the in vivo detection method of the present invention can be used for detection and imaging apoptosis/senescence. Apoptosis/senescence detection may be useful in a variety conditions including monitoring of neurodegenerative conditions, such as Parkinson's disease and amyotrophic lateral sclerosis, as well as giving therapeutic feedback for the efficacy of cancer therapies that rely upon the induction of apoptotic cell death. In a method of detecting apoptsis, optical nanoparticles in accordance with the invention can be conjugated to anti-Amexin V antibodies. Amexin V translocates from the inner cellular membrane to the outer membrane during many forms of apoptotic cell death and has been previously used in nuclear medicine apoptsis detection techniques. The conjugated optical nanoparticles can be administered to a subject and detected and imaged using optical imaging or other imaging techniques (e.g., MRI or CT).

In yet another aspect of the invention, the in vivo detection method of the present invention can be used for the detection, imaging, and localization of neurological pain pathways. In the method, optical nanoparticles (e.g., quantum dots) can be conjugated to neuro-ablative peptides and antibodies to substance P to help identify neurons involved in pain pathways. The conjugated optical nanoparticles can be administered to a subject and detected and imaged using optical imaging or other imaging techniques (e.g., MRI or CT). Identification of the neuroanatomic substrates of pain can allow not only more accurate development of treatment strategies that rely upon advances in pharmacology, but also the modulation of pain signals using newly designed stimulators for use in the central and peripheral nervous system.

In a further aspect of the invention, the in vivo detection method of the present invention can be used for the detection, imaging, and localization of epilepsy foci. In the method, optical nanoparticle (e.g., quantum dots) conjugates (e.g., antibody quantum dot conjugates) to GABA receptors or ion channels over-represented in epileptogenic tissues can be administered to a subject and detected and imaged using optical imaging or other imaging techniques (e.g., MRI or CT).

In another aspect of the invention, the in vivo detection method of the present invention can be used for the detection, imaging, and localization of therapeutic molecules, such as protein immunotoxin conjugates, antibodies, or nucleic acids can also be labeled with optical nanoparticles to be tracked through the body to their target sites. For example, protein immunotoxin labeled with quantum dots can be delivered to brain tumors using convection enhanced delivery (CED). Optical spectroscopy probes can be placed at the periphery of target distribution. Detection of fluorophores will allow the optics systems to detect drug concentration at site of the probe.

In yet another aspect of the invention, the in vivo detection method of the present invention can be used for pharmacokinetics and pharmacodynamics of therapeutic molecules. Therapeutic agents, such as protein, nucleic acid or other large molecules can be labeled with quantum dots for pharmacokinetics and pharmacodynamics studies. Finger probes designed similar to the finger clip of pulse oximetry units used to monitor oxygen concentration during anesthetic procedures can be used for optical spectroscopy monitoring. Using finger probes designed for optical spectroscopy, the circulating concentrations of the therapeutic agents can be monitored based upon the fluorescence signal of the labeled optical nanoparticles. This technique can allow pharmacokinetics and pharmacodynamics studies to be performed without invasive blood draws.

In a further aspect of the invention, the in vivo detection method of the present invention can be used for imaging subjects with renal insufficiency or contrast dye allergies. In the method, a quantum dot shell can be loaded with an electron dense material. Preliminary data using water soluble quantum dots has demonstrated their ability to be imaged in micromolar concentrations by computed tomography. There is no known cross reactivity with iodine and this should lead to the ability to use water soluble quantum dots in multiple x-ray imaging techniques.

Besides the in vivo techniques described above, the present invention relates to the use of optical nanoparticles in rapid detection systems that can be used by primary care level practitioners and field workers in the detection of infectious disease and cancer. These systems can be flexible and allow the detection of a variety of illnesses with portable, inexpensive and easy to use tools. Optical nanoparticle detection mixtures can be designed for detection of a variety of conditions, and require only small amounts of optical nanoparticles in the mixture for each analysis, formulated in a manner to allow long term storage at room temperate.

In one aspect, the present invention relates to the use of optical nanoparticles, such as quantum dots, in the development of rapid diagnostic kits that can be developed for small hospitals, doctor's offices, or field work for the detection of cancer and infectious disease from liquid sources or solid sources, among others. These kits and detection modules can allow primary care level and field level workers, such as physicians, nurses, and aid workers to screen and detect a variety of conditions, leading to early detection and more prompt treatment that otherwise may not be possible. After rigorously tested and compared with standard hospital laboratory techniques for accuracy, these kits can be used in doctor's offices, or in the filed, which would broaden their utility as well as make rapid diagnostics accessible to patients throughout the world.

The present invention, in another aspect, relates to a method of in vivo detection or tracking of therapeutic cells or genes. In the method, genes or therapeutic cells, including stem cells, package delivery cells carrying a packaged gene or protein, and immunotherapeutic cells, with these optical nanoparticles can be labeled ex vivo for tracking and detection in vivo with a variety of imaging techniques. The method includes the detection and therapeutic use of an optical nanoparticle, such as quantum dot or an ultrasmall paramagnetic iron oxide particle (USPIO) for detecting the localization and delivery of cells used in immunotherapy or genes used in gene therapy to their target location using a variety of imaging methods. These imaging methods can include optical imaging and spectroscopy with quantum dots, computed tomography (CT) with quantum dots, or magnetic resonance imaging (MRI) with USPIOs. Use of quantum dots or USPIOs to detect the localization and delivery of gene for therapy and therapeutic cells including stem cells and/or immune cells in these therapeutic modalities is not invasive.

One aspect of the invention relates to a method of using optical nanoparticle (e.g., quantum dot) based rapid screening tests for the detection of urinary track infection. In the method, a plurality of optical nanoparticles with a specific emission spectrum or color can be conjugated to an antibody that is specific for a common urinary pathogen to form a optical nanoparticle-antibody combination. A mixture of pluralities of optical nanoparticles conjugated to antibodies versus multiple common urinary pathogens is prepared to form optical nanoparticle-antibody combinations where each optical nanoparticle-antibody combination is specific for a common urinary pathogen and is encoded with a different color. A urine sample (e.g., about 1 ml) can be obtained from a patient. The optical nanoparticle-antibody combinations can be added to the urine sample.

After allowing a sufficient amount of time (e.g., about 5 minutes) for the optical nanoparticle-antibody combinations to bind to their targeted pathogens in the urine sample, the sample can be loaded onto a spin column. The spin column is a tube that has a filter at one end of the tube that holds the sample inside the tube. The pore diameter of the filter can be, for example, about 20 to about 100 nm. A mini-centrifuge can be used to spin the column loaded with the sample. Optical nanoparticle-antibody combinations that didn't bind to pathogens will flow through the filter together with other substances in the sample that have sizes less than 20 nm.

Because optical nanoparticle-antibody combinations bound to pathogens are bigger than 100 nm, they will be retained on the filter. An optical spectroscopic system or long-pass filter system can be used to view the filter by applying a blue or UV light source to the filter. Any pathogen bound to optical nanoparticle antibody combination will glow in the color of the quantum dot-antibody combination. The detection of the color of a specific optical nanoparticle-antibody combination on the filter suggests the detection of its targeted pathogen from the urinary sample. Once the pathogen or pathogens are identified from the urinary sample, the nature of the urinary infection can then be defined accordingly.

In another aspect of the invention, optical nanoparticles, such as quantum dots, can be used in a method to detect parasite ova antigens in a stool sample. In the method, a plurality of optical nanoparticles with a specific emission spectrum or color are conjugated to an antibody that is specific for a common stool parasite pathogen to form an optical nanoparticle-antibody combination. A mixture of pluralities of optical nanoparticles conjugated to antibodies versus multiple common stool parasite pathogens is prepared to form optical nanoparticle-antibody combinations where each optical nanoparticle-antibody combination is specific for a common stool parasite pathogen and is encoded with a different color. A stool sample can then be collected using, for example, a swab. The swab can then immersed in a quantity (e.g., about 5 ml) of a saline-optical nanoparticle-antibody combination mixture.

After allowing a sufficient amount of time (e.g., about 55 minutes) for the optical nanoparticle-antibody combinations to bind to their targeted pathogens in the stool sample on the swab, the swab can be removed and washed (e.g., about 5 times in about 5 ml saline for about 1 minute each) to remove optical nanoparticle-antibody combinations that did not bind. An optical spectroscopic system or long-pass filter system can be used to view the swab by applying a blue or UV light source. Any parasite antigens on the swab will bind to its specific optical nanoparticle-antibody combination and glow in the color of the quantum dot-antibody combinations. The detection of the color of a specific optical nanoparticle-antibody combination on the swab suggests the detection of its targeted parasite pathogen from the stool sample. Once the parasite pathogen or pathogens are identified from the stool sample, the nature of the infection can then be defined accordingly.

It will be appreciated, that using similar techniques and processes described in cancer detection methods, nipple discharge or aspirates can be used for ex vivo detection of breast cancer cells nucleic acid sequences or antigens; stool samples can be used for gastrointestinal (GI) cancer detection; urine samples can be screened for renal cell carcinoma, bladder and prostate cancer; semen can be screened for prostate cancer; and mucous can be screened for aerodigestive or lung cancer. Additionally, any antigens or nucleic acid sequences diagnostic of disease can be used in a quantum dots based rapid screening test similar to that described above for infectious disease detection.

### EXAMPLES

### Example 1

Optical Spectroscopy and Quantum Dots

The most common initial therapy for brain tumors is surgical resection, which can benefit the patient several ways. This procedure relieves the mass effect of tumor on neurological tissue and allows histological diagnosis of the tumor, which directly affects the direction of follow-p therapeutic strategy. Many studies have demonstrated that the degree of resection significantly influences the survival length and quality of life for brain tumor patients. The goal of surgical resection, therefore, is to remove the maximum amount of tumor mass without sacrificing the patient's neurologic function. To achieve this goal, accurate intraoperative identification of brain tumor margins during craniotomy is required.

Quantum dots with a wavelength of 705 nm allow for further distinction from normal tissue and tumor tissue through injection in the patients bloodstream. The neurosurgeon will then be able to have a “bull's eye” viewing of the tumor for easier extraction.

In past investigations, fluorescence dyes were used to enhance brain tumor detection, such as 5-aminoleveulinc. However, low sensitivity of this method has been reported, as the fluorescence dye is not taken up by tumor cells where the blocked brain barrier interact.

Our theory proposes quantum dots will co-localize to the brain tumor through the macrophages of the brain tissue, allowing an outline to form and the tumor to be completely extracted. The quantum dots will be injected directly into the bloodstream and will be filtered out of the bloodstream through the liver and spleen and once liver and spleen are full the quantum dots will travel to the brain and co-localize to the macrophages. With an advanced fluorescence probe, we can detect a distinct 705 peak, and accurately locate the tissue. In this study we will show how fluorescence of quantum dots can be detected from tissue and their detection intensity in solution.

Materials and Methods

Materials

The principal material used in this experiment was quantum dots 705 nm (Quantum dot Corp. Hayward, Calif.). Other materials include PBS, 4% paraformaldehyde, brain, liver and spleen taken from rats (Fisher rats, Charles River NCI, Wilmington, Mass.). Cell lines injected in the rats were C6 glioblastoma tumor cells, and C6 with GFP (green fluorescence protein).

Instrumentation

Referring to FIG. 6, which is a schematic illustration of an optical detection system, optical spectra of quantum dots were collected through the detection of wavelength (nm) after excitement by a nitrogen laser. This high-pressure nitrogen laser (337 nm, Oriel Corporation, Stratford, Conn.) emitted a blue light and created a fluorescence to cause the response. A 150 W illuminator (Fiber Lite, Model 180, Edmund Scientific Company Tonawanda, N.Y.) emitting broadband white light from wavelengths or 400-800 nm was used for diffuse reflectance measurements. We also used two probes, a larger surface probe developed for testing surfaces and a 25-gauge needle probe developed by MK Optics INC. (Chesterland Ohio) to test inside tissues. Both probes used in the detection contain three fibers. Directed conventionally is the center fiber, the tip of the fiber is tapered to optimize the excitation radius. The other two fibers surround this “collection” fiber, pulse a laser and shine a white light respectively. With this design it allows the fibers to extract light and collect data simultaneously producing an accurate representation of the sample. The fibers are all twined with in a stainless steel coating for easy optical practicality. This allows the user to bring it in the operating room as necessary and protects the actual fibers from breaking, cracking or bending. Three separate tubes connect and then are fused together to allow for the probes' easy maneuverability.

The signal was collected and dispersed using a spectrometer (S2000-FL, Ocean Optics, Dunedin, Fla.) and collected from the spectrometer into a controller, which is directly connected to a laptop computer (Latitude D600 Dell laptop, Round Rock, Tex.), which sorted the data using a Labview, (National Instruments corporation Austin, Tex.), Matlab, (Mathworks Inc., Natick, Mass.) and Excel, (Microsoft Corporation, Redmond, Wash.) collaboration. The Labview program used was written by Wei-Chang Lin, Vanderbilt University (C 2003, and is a useful tool to interface the probe with the computer to obtain real-world results and analyze the data to share results in with Matlab for easy analysis. The entire system runs using the computer and all of its programs.

Experimental Methods

Since the entire system is controlled using a laptop, the system becomes very user friendly. It allows the user to hold the probe against the desired sample and acquire on the computer screen. When using the larger surface probe, the quantum dots in solution were placed in a quartz cuvette, tested using various integration times, the time the laser and light are on, and using different concentration of quantum dots in a water solution.

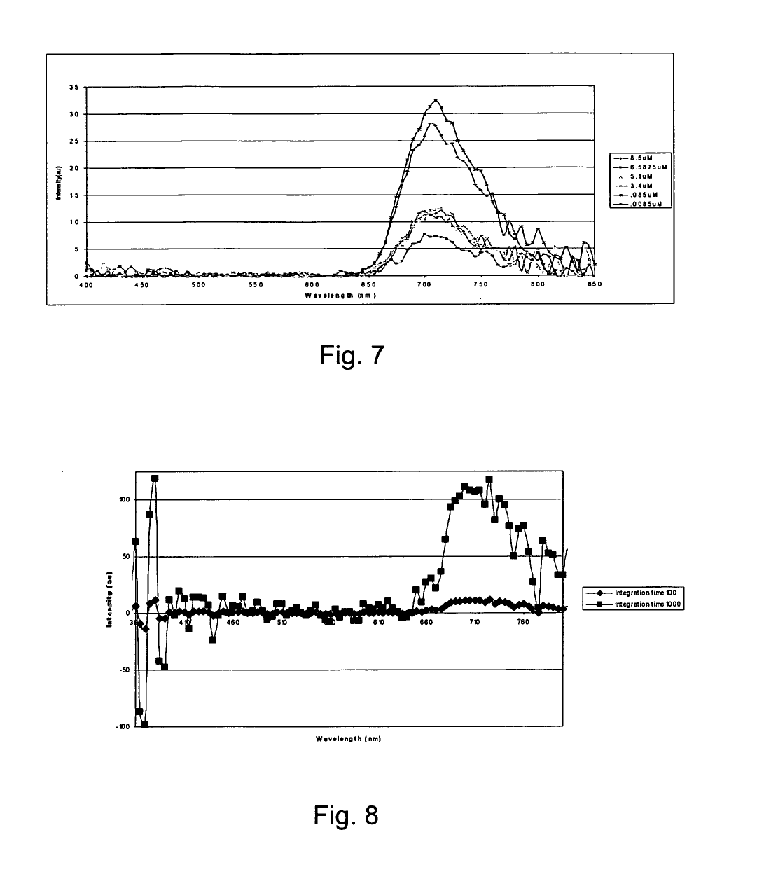

When using the needle probe the process is a bit more complex. The probe will be placed directly into the quantum dot solution and the readings are taken. The system then was attached to an optics bench and placed in such a way that the fibers of the 25-gauge probe would be stationary. Once set up, the tissue was placed in a small dish located directly below the needle and rose until the needle was just penetrating the tissue. Readings were then taken and the results are provided in FIG. 7.

Data Analysis

Spectral data were post-processed before any analysis was conducted. Background subtraction was first preformed on each spectrum with its corresponding baseline measurement. Correction factors (C) were generated by taking ratios between the standard spectra [S(λ)] measured at the start of the study and those acquired for very experiment of the study.
Cᵢ=Sᵢ(λ)/(Sₗ λ), (1)
Where S(λ)=F_{ref}(λ) or Rd_{ref}(λ), λ=620 nm for fluorescence, 700 nm for reflectance, i=1 to n, is the total number of experiments. For each stage the data that is acquired is stored into a temporary data file that is used by Matlab corresponding to the i as the initial experiment to n/3, the total number of experiments run. Each correction factor Cᵢ was then to every sample spectrum acquired in a given experiment i, to ensure that spectral intensity is a valid discrimination of information.

The analysis of the data took place after the excitation and acquisition wavelengths were predetermined, so that verification of the ability of the machine could take place. We noticed different sizes of peaks, intensity and over all shape changed based on what we tested the quantum dots in, how long the integration time was set and the background light setting used.

Results

The diagrams below show the different collected data. In FIG. 7, we tested various concentrations, and in FIG. 8 changed the integration time. As you can see there is a direct relationship with the data that we collected and the conditions on the solution. All of these experiments were preformed in a dark room with only a red photo lamp lit.

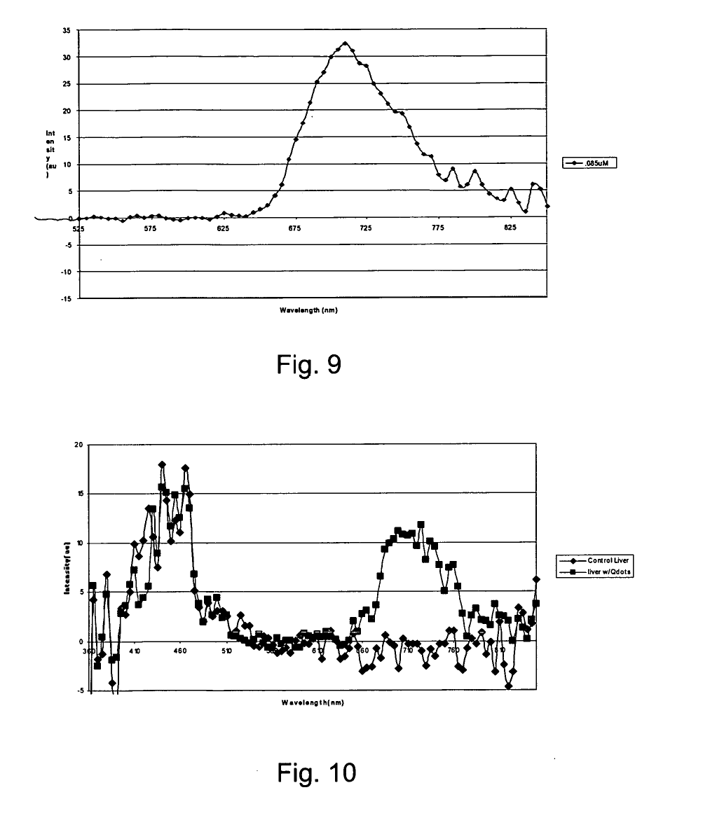

When running the experiments the set-up of the system allows the user to hold the probe while seated. Thus, it will minimize movement between the cuvette and the probe during each experimental test. This method is very useful when the integration time increases because the longer the light is exciting the sample, the more important it becomes to have everything as still as possible to record accurate data. The experiments are preformed in the dark room to minimize the sound to noise ratio of the surrounding also providing a more accurate reading. In FIG. 9 we tested a 0.085 μM solution of quantum dots in the dark room and the background light drastically faded. After using the surface probe to test quantum dots in solution we then wanted to track quantum dots in tissues. Using the needle probe we tested a control liver and a liver with quantum dots, shown in FIG. 10. The NADPH peak is present in both and the 705 nm peak from the liver containing quantum dots. The integration time, when using the needle probe, is about 10× longer because the collection fiber has a smaller radius to collect light.

### Example 2

Quantum Dots are Phagocytized by Macrophages and Co-Localize with Experimental Glioma

Quantum dots are optical nanoparticles constructed of an optical core, surrounded by a protective shell. The shell may be modified to promote water solubility, allow conjugation to proteins and nucleic acids, and to improve quantum dots circulation half-life. Previous studies have shown that the circulation half-life of quantum dots might be prolonged by increasing the length of polyethylene glycol (PEG) polymers attached to the quantum dot shell. We hypothesized that intravenous injection of quantum dots with long PEG group surface modifications would improve the circulation half-life of quantum dots and diminish non-specific uptake by the RES, thereby enhancing phagocytosis by tumor infiltrating macrophages, creating a technique to optically visualize brain tumors in vivo.

Water soluble cadmium/selenide (Cd/Se) core, zinc sulfide (Zn/S) shell quantum dots emitting at 705 nm had their shells conjugated with methoxy-terminated PEG amine of 5000 Daltons. C6 rat gliosarcoma cell lines were stereotactically implanted into the in right frontal lobe of Fisher rats (n=10). Two weeks after tumor implantation, MRIs were performed to confirm tumor growth (Tumor size 2-8 mm). Animals were injected intravenously with 3.4, 8.5, or 17 nanomoles of quantum dots. Control animals were given the highest dose of quantum dots without tumor implantation (n=3). Animals were sacrificed 24 to 48 hours after injection and their tissues analyzed with optical imaging and histopathological techniques.

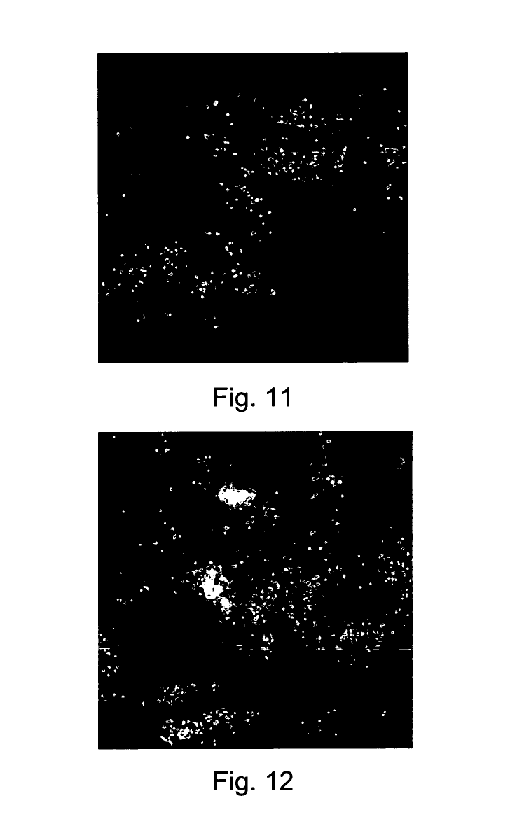

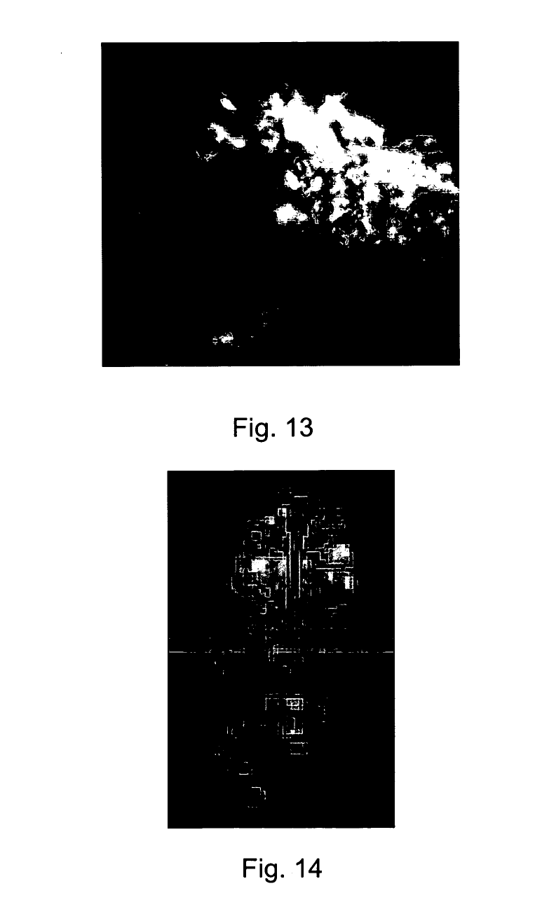

A dose response relationship was observed between the amount of quantum dots injected and the concentration of quantum dots in experimental glioma. At 3.4 nanomole injections of quantum dots, liver, spleen, and lymph nodes had large concentrations of quantum dots (FIG. 11-13), but only rare quantum dots were identified in the rat brain tumors. At 8.5 nanomole, more frequent quantum dots were visible in experimental gliomas by high power UV microscopy (FIG. 2), but the number of quantum dots remained below the detection threshold of optical imaging techniques in thick, low-power tissue slices or at the whole brain level.

Further increases in the injected dose of quantum dots to 17 nanomoles had no apparent acute toxicity on the animals during the course of the experiments. There was no evidence of injury or inflammation in any of the brain, liver, spleen, or lymph nodal tissues studied microscopically. Microscopic examination of RES tissues showed a small increase in the quantum dots concentration in these tissues between 3.4 and 8.5 nM injection. However, there was no significant difference in the concentration of quantum dots in the RES between 8.5 and 17 nM. This suggests that maximal RES phagocytosis of quantum dots was reached between 3.4 and 8.5 nM injection and prior to the large increases in the number of quantum dots visualized in brain tumors themselves at 17 nM quantum dot injection

TABLE 1

| | | Mean QDot | | |
|---|---|---|---|---|
| | QDots nM | counts/HPF | SD | P value |
| Brain | 3.4 | 0.3 | 0.7 | P = 0.001* |
| | 8.5 | 8.0 | 54 | P = 0.000045** |
| | 17 | 114.4 | 45.3 | |
| Liver | 3.4 | 126.8 | 19.0 | P = 0.028# |
| | 8.5 | 178.4 | 16.7 | P = 0.50## |
| | 17 | 167.8 | 28.5 | |

*Comparison of 3.4 nM vs. 8.5 nM injection.
**Comparison of 8.5 nM vs. 17 nM injection.
#Comparison of 3.4 nM vs. 8.5 nM injection.
##Comparison of 8.5 nM vs. 17 nM injection.

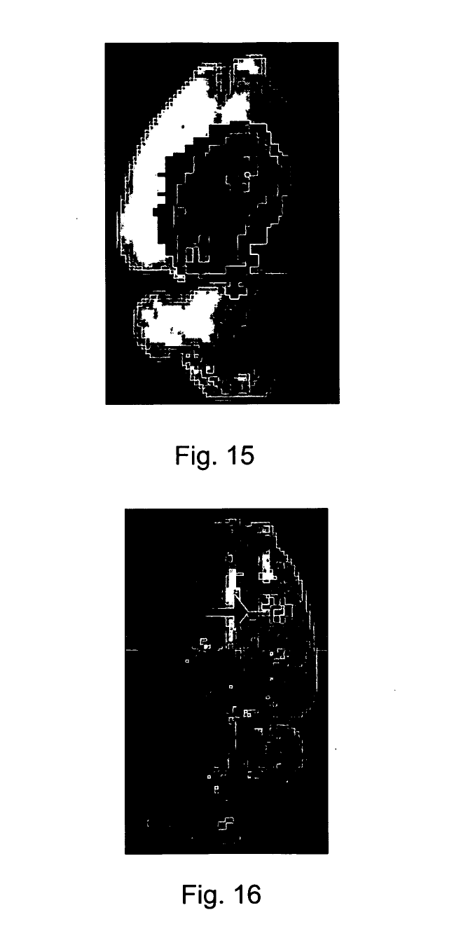

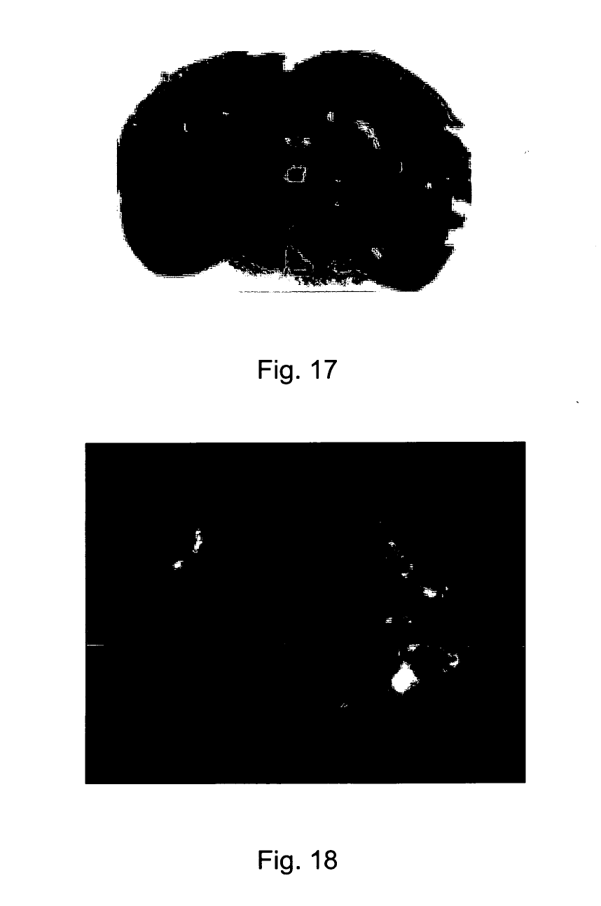

At the highest dose studied, optical imaging techniques identified QD-specific fluorescence within the experimental gliomas at the whole brain and tissue slice levels (FIGS. 14-18). In some animals, fluorescence imaging identified satellite tumors distant from the implant site (FIG. 16). H&E stains and UV microscopy were used to confirm that the region of fluorescence contained tumor cells and quantum dots in both the primary tumor implants and within the satellite lesions. This example demonstrates the ability of quantum dot-based optical imaging techniques to outline even small, metastatic lesions.

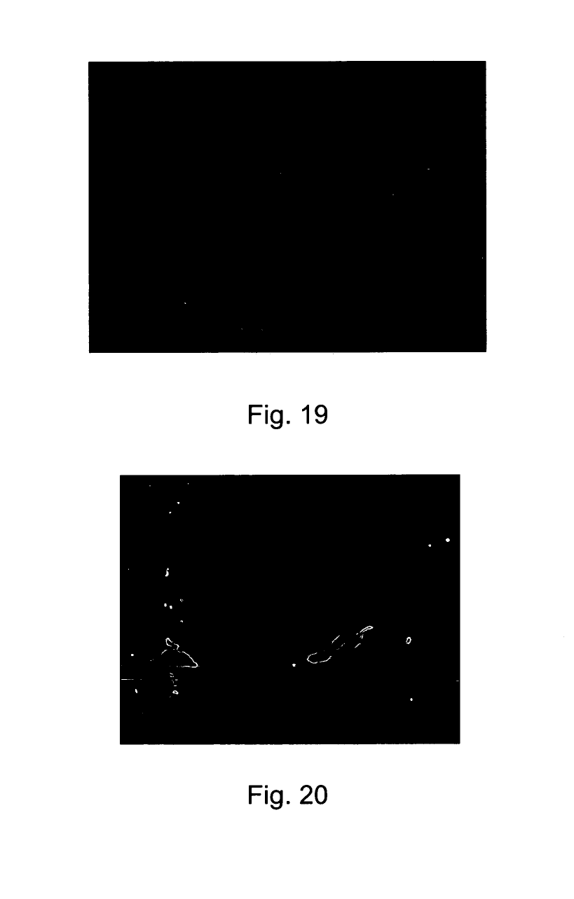

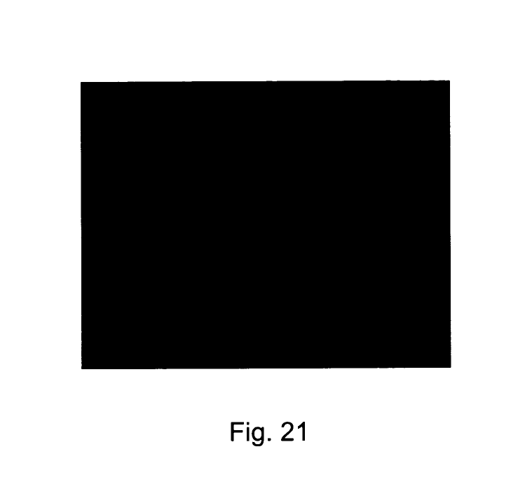

Quantum dots identified within the brains of experimental animals appeared to home specifically to the gliomas. Control, non-tumor-bearing animals given quantum dots had no evidence of quantum dots in brain tissues. Similarly, the “normal” contralateral hemispheres of animals with gliomas had no quantum dots present. Even the normal white matter immediately adjacent to tumor borders was without quantum dots (FIG. 19). Confocal microscopy using glial fibrillary acidic protein (GFAP), an intermediate filament found in the cytoplasm and dendritic processes of normal astrocytes, demonstrated that quantum dots were absent from GFAP positive astrocytes (FIG. 20). The quantum dots were specifically localized to CD11b positive cells, the macrophages and microglia (FIG. 21). Although it is possible that circulating macrophages phagocytize quantum dots and are drawn to the tumor by cytokines, we hypothesize that the dual CD11b/quantum dot positive cells are the resident tumor-infiltrating macrophages and microglia. We believe that PEG-coated quantum dots are able to either evade or saturate the RES, or both, at higher doses. This enhances the exposure of circulating quantum dots to tissue macrophages and microglia in or near the brain tumors, effectively tagging them optically.

In the treatment of human gliomas, patient survival correlates with the extent of resection. Image guided surgery, nearly universally used in the surgery of gliomas, relies upon preoperative images such as MRIs to guide the neurosurgeon during tumor biopsy and resection. Unfortunately, the spatial resolution of the preoperative data degrades during a procedure due to registration inaccuracies and brain tissue shifts during brain tumor resection. Optical techniques, such as optical labeling of tumors with quantum dots, can provide the surgeon with critical data regarding the location of the tumor in real time during tumor resections and biopsies.

Additional modifications to quantum dots, such as coating the quantum dot shell with gadolinium, a paramagnetic MRI contrast agent, appear capable of preserving quantum dot optical properties while adding the ability to detect quantum dots with MRI. A quantum dot conjugated to gadolinium might thereby function as a multimodal imaging particle, detectable by MRI for preoperative surgical navigational studies, and detectable intraoperatively using optical techniques. Such a multimodal imaging particle can provide the surgeon with feedback on the location of tumor and completeness of resection during glioma surgery, improving upon the inherent inaccuracies of current systems of image guided surgery that rely solely upon preoperative imaging.

Optical imaging and optical spectroscopic systems are available to identify quantum dots that emit in the red or near-infrared spectra. Optical detection systems are relatively inexpensive compared to intraoperative techniques such as MRI. Existing optical systems may be adapted easily to open surgical resections of tumor in an attempt to improve surgical resections and patient survival. Moreover, prototype optical systems have been designed that are capable of delivering light through biopsy needles (unpublished data), thus permitting less invasive, more accurate, optically-guided surgical procedures in the brain and elsewhere in the body.

We have demonstrated that the intravenous delivery of PEG-coated quantum dots with long circulation half-lives in nanomolar concentrations can escape sequestration in the liver, spleen and lymph nodes. This allows phagocytic cells such as tumor infiltrating macrophages in other regions of the body to engulf quantum dots, optically outlining the tumor mass. Optical imaging and spectroscopic techniques exist to use this information to guide the completeness of surgical resection of tumors, or to aid in the identification of tumors during needle biopsy, potentially improving patient outcomes in brain tumor surgery.

Methods

Brain Tumor Models

Rats were injected using a rat stereotactic frame (David Kopf Instruments, Tujunga, Calif.) using landmarks of 2.5 mm lateral to bregma and 1 mm anterior to bregma using the ipsilateral basal ganglia as the target. After twist drill craniotomy, 5×10⁵ C6 rat glioma cells (ATCC, Manassas, Va.) were injected using a 27 gauge needle. Rats were housed in negative pressure isolation cages and tumors grown for 14 days prior to treatment.

Magnetic Resonance Imaging (MRI)

Rats were anesthetized with 200 μl of ketamine and taken for MRI. The animals were injected with 200 μl of gadolinium (Berlex Laboratories, Wayne, N.J.) via tail vein injection. After allowing 5 minutes circulation time, T1-weighted images were performed on a 3T MRI (Siemens AG, Erlanger, Germany) unit after positioning the animals within a human wrist coil. Animals in whom tumor growth was confirmed were selected for quantum dot injection.

Quantum Dots

Coreshell zinc sulfide-cadmium selenide quantum dots emitting at 705 nm with a 5000 Dalton amino polyethylene glycol polymer attached to the shell were used in this study (Quantum Dot Corporation, Hayward, Calif.). Quantum dots were injected via rat tail vein method. 3-17 nM of the quantum dots were injected slowly over 2 minutes.

Histopathology

Animals were sacrificed after MRI, 24 to 48 hours post quantum dot injection with CO₂ and potassium chloride intracardiac injection. Brains, livers, spleens, and lymph nodes were fixed in a sucrose solution prior to formalin fixation. Formalin fixed tissues were section at 8 μm using a cryostat (Leica Microsystems AG, Wetzlar, Germany). Tissue sections were stained with hemotoxylin and eosin. Sections for quantum dot visualization were left unstained for ultraviolet (UV) microscopy (Nikon, Melville, N.Y.).

Immunohistochemistry was performed with primary antibodies to GFAP (Sigma-Aldrich, Cambridge, Mass.), CD11b (Oxford, UK), and secondary antibodies of FITC (Sigma-Aldrich ) and 525 nm-emitting quantum dot secondary Antibodies (Quantum Dot Corporation).

Fluorescence Imaging

Quantum dots were visualized in whole organs using a fluorescence imaging chamber (Xenogen IVIS system, filter set number three, Alameda, Calif.). Stereoscopic images of thick tissue slices were imaged with a stereoscope (Leica) using a filter set optimized for 705 nm quantum dots (400 nm±50 nm excitation; 705 nm±20 nm emission) (Chroma Technology Corporation, Rockingham, Vt.). UV photo microscopy was performed on a microscope at 200× (Nikon) and images collected using a camera system (Polaroid, Waltham, Mass.) prior to digital storage (Dell Computers, Round Rock, Tex.).

Statistics

Quantum dot counts per high power filed were recorded from livers and brains of 2 animals at each dose. Data was entered in Sigma Plot 2001 (Systat Software, Inc., Point Richmond, Calif.). Means, standard deviations, and paired t-tests were used to analyze the data.

From the above description of the invention, those skilled in the art will perceive improvements, changes and modifications. Such improvements, changes and modifications within the skill of the art are intended to be covered by the appended claims.

Having described the invention, the following is claimed:

1. A method of identifying cells of a living subject in vivo, the method comprising:
delivering a plurality of optical nanoparticles to the cells;
optically imaging the optical nanoparticles; and
identifying the cells of a living subject from an image and/or spectrum of the optical nanoparticles.

2. The method of claim 1, the optically imaging of the optical nanoparticles comprising:
illuminating the optical nanoparticles with an excitation light;
collecting light returned from the illuminated quantum dot; and
obtaining an image and/or spectrum of the quantum dot from the collected light.

3. The method of claim 1, the optical nanoparticles comprising quantum dots.

4. The method of claim 3, the quantum dots being water soluble.

5. The method of claim 1, the optical nanoparticles being conjugated to at least one of nucleic acids, proteins, peptides, and antibodies.

6. The method of claim 1, the optical nanoparticles being delivered to the cells by intravenous infusion of the optical nanoparticles into the subject being treated.

7. The method of claim 6, the optical nanoparticles being infused into the subject at a concentration effective to emit a fluorescence emission wavelength sufficient for detecting and imaging the cells when excited with an appropriate excitation wavelength.

8. A surgical method comprising:
preoperatively delivering an effective amount of optical nanoparticles to a target tissue;
imaging the optical nanoparticles; and
guiding a surgical procedure on the target tissue using the images of the optical nanoparticles.

9. The method of claim 8, the target tissue being at least one of neoplastic or inflamed tissue.

10. The method of claim 8, the target tissue comprising a brain tumor.

11. The method of claim 8, the target tissue comprising abnormal tissues.

12. The method of claim 8, the surgical method comprising at least one of a biopsy, laser ablation, radiofrequency ablation, excision, or resection.

13. The method of claim 8, the imaging of the optical nanoparticles being performed preoperatively.

14. The method of claim 8, the imaging of the optical nanoparticles being performed intra-operatively.

15. The method of claim 8, the optical nanoparticles comprising a plurality of quantum dots.

16. The method of claim 8, the imaging of the optical nanoparticles being performed utilizing at least one of computed tomography, magnetic resonance imaging, and optical imaging.

17. A method of identifying a cause for a medical condition of a living subject, the method comprising:
conjugating a plurality of optical nanoparticles with an antibody responsive to the medical condition;
delivering an effective amount of the optical nanoparticles with the antibody to the circulation system of the living subject;
optically imaging the optical nanoparticles; and
determining the cause for the medical condition from the images of the optical nanoparticles conjugated with the antibody.

18. The method of claim 17, the plurality of optical nanoparticles comprising quantum dots with different wavelengths, and the antibody conjugated with quantum dots with a predetermined wavelength.

19. The method of claim 18, the images of the quantum dots conjugated with the antibody comprise a spectrum with corresponding wavelength quantum dots.

20. The method of claim 19, the determining step comprising a step of identifying the predetermined wavelength from the spectrum so as to determine the organism for the medical condition.

21. A method of using quantum dots as contrast agent for imaging at least a part of a living subject, the method comprising:
loading an electron dense material into a quantum dot shell;
delivering an effective amount of quantum dots loaded with the electron dense material to the part of the living subject;
optically imaging the part of the living subject; and
obtaining medical information regarding the living subject from the optical images of the living subject.

22. A method of detection of a substance in a sample, the method comprising:
adding a plurality of optical nanoparticles to the sample;
waiting for a period of time to allow the plurality of optical nanoparticles to bind to the substance in the sample to form a optical nanoparticle dot-substance mixture;
removing optical nanoparticles that do not bind to the substance in the sample;
illuminating the optical nanoparticle-substance mixture in the sample with an excitation light;
collecting light returned from the illuminated optical nanoparticle-substance mixture in the sample;
obtaining an image and/or spectrum of the optical nanoparticle-substance mixture in the sample from the collected light; and
detecting the substance from the image and/or spectrum of the optical nanoparticle-substance mixture.

23. The method of claim 22, further comprising conjugating the plurality of optical nanoparticles with a predetermined chemical compound.

24. The method of claim 22, wherein the predetermined chemical compound comprises at least one of a peptide, protein, nucleic acid, or a antibody.

25. The method of claim 22, further comprising the steps of finding a sign of pathogenesis from the image and/or spectrum of the optical nanoparticle-substance mixture in the sample and identifying a corresponding disease from the identified sign of pathogenesis from the plurality of quantum dots with a predetermined chemical compound.

26. An intraoperative guidance system comprising:
a plurality of optical nanoparticles provided in a subject; and
an optical device capable of being positioned in the subject for detection of the optical nanoparticles.

27. The intraoperative guidance system of claim 26, the optical nanoparticles comprising a plurality of water soluble quantum dots.

28. The intraoperative guidance system of claim 27, the optical device comprising an optical probe, the optical probe including a light delivery and a light collection means.

29. The intraoperative guidance system of claim 26, the light delivery means being capable of emitting light at a first wavelength effective to excite the optical nanoparticles and light collection means being capable of collecting light emitted upon excitation of the optical nanoparticles.

30. The intraoperative guidance system of claim 26, including a means for imaging the detected optical nanoparticles.

31. The intraoperative guidance system of claim 26, the optical device transmitting light at a first wavelength and collecting light at a second wavelength.
